(19) United States
(12) Patent Application Publication
(10) Pub. No.: US 2011/0318431 A1
(43) Pub. Date: Dec. 29, 2011

(54) Pharmaceutical Compositions and Methods of Treating Neurological Insults

(75) Inventor: Anil Gulati, Naperville, IL (US)

(73) Assignee: ENDOGENX, Los Gatos, CA (US)

(21) Appl. No.: 13/142,675

(22) PCT Filed: Dec. 29, 2009
(86) PCT No.: PCT/US09/69630

§ 371 Date: Sep. 14, 2011

Related U.S. Application Data
(60) Provisional application No. 61/141,302, filed on Dec. 30, 2008.

(51) Int. Cl. A61K 33/14; A61P 25/00; A61P 1/16; A61P 25/06; A61P 3/10; A61P 9/10; A61P 25/08; A61K 33/06; A61P 35/00

(52) U.S. Cl. 424/681; 424/682

(57) ABSTRACT

A pharmaceutical composition containing a magnesium salt and an osmotic hypertonic agent, like a mannitol, is disclosed. Also disclosed are methods of treating individuals who have suffered a neurological insult, such as traumatic brain injury.

### CROSS-REFERENCE TO RELATED APPLICATIONS

This application claims the benefit of U.S. provisional patent Application No. 61/141,302, filed Dec. 30, 2008, incorporated herein by reference in its entirety.

### FIELD OF THE INVENTION

The present invention relates to pharmaceutical compositions comprising (a) a magnesium salt and (b) a hypertonic osmotic agent, such as mannitol or hypertonic saline, and to administering a magnesium salt and a hypertonic osmotic agent in methods of treating individuals who have suffered a neurological insult.

### BACKGROUND OF THE INVENTION

Magnesium is the fourth most available cation in the body, and the second most abundant cation in the intracellular fluid. Its presence in the extracellular fluid is important because of its homeostatic and physiological role. Magnesium is a vital nutrient, and is important for normal cellular functions as a cofactor for more than 325 enzymes, glycolysis, Krebs cycle activity, oxidative phosphorylation, and for maintaining membrane integrity.

Magnesium is involved in a number of bioenergetic and biochemical activities, and plays an important role in normal neuronal activity. Magnesium also has a significant role in protein synthesis, membrane stability and fluidity, RNA aggregation, and is a cofactor in DNA and protein synthesis. Additionally, magnesium is important for neuromuscular and smooth muscle tone, regulation of calcium transport, and reduction of calcium accumulation.

Magnesium is found mostly in bone (53%), soft tissues (46%), and blood (1%). Magnesium in the blood is found in three major forms: 27-34% bound to proteins; 8-12% complexed to inorganic or organic anions; and 54-65% as ionized or free form. The free, ionized form is the physiologically active form of magnesium. The normal range of total serum magnesium concentration is 2.4-3.2 mg/dL, the ionized serum magnesium concentration is 1.2-2.3 mg/dL, and the total cerebrospinal fluid (CSF) magnesium concentration is 1.1-1.6 mg/dL.

Polyhydroxy monosaccharides include such common sugar alcohols as glycol, glycerol, erythritol, threitol, arabitol, xylitol, ribitol, mannitol, sorbitol, dulcitol, and iditol. Mannitol has been widely used as a hypertonic osmotic agent to treat brain edema and to decrease intracranial pressure in a treatment known as mannitol osmothcrapy. Mannitol does not cross the blood-brain harrier (BBB), but rather acts by drawing water from the interstitial and intracellular spaces of the brain across the BBB. The osmotic action of hyperosmolar mannitol confuses the BBB and delivers drugs to the brain.

To disrupt the BBB, mannitol is administered by the intracarotid route. Shrinking the endothelial cells of the blood vessel walls causes osmotic stress which in turn compromises the integrity of the BBB. For experimental and therapeutic purposes, this procedure is widely used to facilitate entry of water soluble drugs, proteins, diagnostic agents, and other xenobiotics into the brain, which otherwise could not enter the brain. The process is reversible, and the major effects primarily are limited to the BBB. In the brain, mannitol is confined to the extracellular space. The osmotic effect is rapid (in minutes) and lasts for a short period of time because it is rapidly excreted unchanged by the kidneys.

Traumatic brain injury (TBI) and its subsequent cascade of events continue to be a major economic burden to society in spite of vast technological and medical advances in neurosurgery and neurocritical care. Despite having a trauma response system, increased diagnosis of and therapy for secondary injury, and following the “Guidelines of Head Injury”, improvements in mortality, but not morbidity, of TBI patients have been achieved. The neurological benefits remain suboptimal. Most TBI patients undergo neurosurgery to repair hematomas (ruptured blood vessels) and contusions. Some common long term disabilities are stupor, coma, vegetative state and failures of cognition (thinking, memory, reasoning), sensory feelings (sight, sound, touch, taste, smell), communication (expression, understanding), and behavior (depression. anxiety, personality changes. aggression). Some patients also develop other medical complications, like epilepsy, hydrocephalus, cerebral spinal fluid leaks, infections, vascular injuries, cranial nerve injuries, pain, bed sores, multiple organ failure, and poly-trauma.

In a military context, a signature injury among military personnel is TBI resulting from explosive blasts. Soldiers often wear KEVLAR® body armor and helmets as protection from bullets and shrapnel, thus reducing penetrating brain injuries and improving morbidity and mortality. During a blast, however, the human brain can be injured by objects in motion (secondary blast injury) or by an individual being forcefully put into motion by the blast (tertiary blast injury). KEVLAR® helmets do not protect soldiers from blast-induced closed head injuries that are more difficult to diagnose than penetrating TBIs. Closed head injuries include diffuse axonal injury, contusion, and subdural hemorrhage. Diffuse axonal injuries occur when shearing, stretching, and/or angular forces pull on axons and small vessels, leading to axonal swelling and disconnection. Contusion occurs when the brain moves within the skull or strikes the skull leading to hemorrhage and edema. Traumatic subdural hemorrhage occurs when the brain impacts or strikes the skull with sufficient force to injure the tributary surface veins. Such secondary brain insults adversely affect clinical outcome in patients with brain injury.

Depletion of magnesium is observed in animal brains and human blood after a brain injury. It has been found that treatment with magnesium attenuates the pathological and behavioral changes in rats with brain injury. Systematic administration studies in rats have shown that magnesium enters the brain, and treatment with magnesium following brain cortical injury in rats has been shown to be neuroprotective. However, the therapeutic effect of magnesium has not been consistently observed in humans with traumatic brain injury (TBI). Inducing hypermagnesemia in humans did not concomitantly increase magnesium levels in the CSF. In contrast to preclinical studies on rats, clinical studies on humans using magnesium alone following TBI failed to show any beneficial effects. Accordingly, an unsolved need exists in the art for the effective treatment of individuals who have suffered a neurological insult.

### SUMMARY OF THE INVENTION

Mannitol (20%) is used as a diuretic, reduce brain edema, and disrupt the blood brain barrier to allow the entry of drugs into the brain. Magnesium also has demonstrated a significant neuroprotective effect. However, magnesium has side effects on the heart that limit its use. The present invention relates to compositions containing a magnesium salt and a hypertonic osmotic agent, and to methods of using this combination for neuroprotection. The hypertonic osmotic agent opens the blood brain barrier (BBB), enhances the transport of magnesium into the brain, and increases the neuroprotective effect of magnesium.

The present invention therefore is directed to pharmaceutical compositions comprising (a) a magnesium salt and (b) a hypertonic osmotic agent, like mannitol. The compositions also can contain excipients and/or pharmaceutically acceptable carriers. In another embodiment, the present compositions are administered, or a magnesium salt and a hypertonic osmotic agent are administered from separate compositions, in methods of treating individuals who have suffered a neurological insult. The present method treats traumatic brain injury, as well as various neurological disorders, by the administration of a therapeutically effective amount of a magnesium salt and a hypertonic osmotic agent to an individual in need thereof.

In some embodiments, the magnesium salt comprises magnesium sulfate, magnesium chloride, or a mixture thereof. In other embodiments, the hypertonic osmotic agent comprises mannitol, hypertonic saline, or a mixture thereof. In various embodiments, the weight ratio of magnesium salt to hypertonic osmotic agent is about 1:0.1 to about 1:100. In still other embodiments, the composition consists essentially of a magnesium salt and mannitol.

In various embodiments, the present invention is directed to methods of treating traumatic brain injury, as well as disorders associated with increased intracranial pressure, brain edema due to head injury, intoxications, hepatic failure, space-occupying cerebral lesions, meningitis, Reye's syndrome, cerebral malaria, brain tumors, birth asphyxia, perinatal asphyxia, asphyxiated neonates and infants, rebound phenomenon in the treatment of raised intracranial pressure, hyperglycemic crisis and its complications, diabetic ketoacidosis, acute stroke, ischemic stroke, cerebral hemorrhage, focal ischemia, subarachnoid hemorrhage, drug induced hypomagnesaemia, neurological complications of chemotherapeutic agents, preeclampsia, epileptic episodes, prolongation of opiate and non-opiate analgesia, affective disorders, post-traumatic depression/anxiety, neuropsychiatric disorders, headaches and migraines, and neuroprotection of the adult and neonatal brain.

A composition of the present invention can be administered (or a magnesium salt and a hypertonic osmotic agent can be administered from separate compositions) in the treatment of a neurological insult, or in conjunction with an additional therapy useful in a treatment for the neurological insult. The additional therapy can be one or both of a pharmacological treatment and a physiological treatment. The pharmacological treatment can be, for example, administration of dexanabinol, progesterone, or both. The physiological treatment can be hypothermia, hyperoxia, or both.

The magnesium salt, hypertonic osmotic agent, and additional therapeutic agent can be administered together as a single-unit dose or separately as multi-unit doses, wherein the present composition is administered before the additional therapeutic agent or vice versa. It is envisioned that one or more dose of a present composition and/or one or more dose of an additional therapeutic agent can be administered.

In one embodiment, a present composition and an additional therapeutic agent are administered simultaneously. In related embodiments, a present composition and the additional therapeutic agent are administered from a single composition or from separate compositions. In a further embodiment, the present composition and additional therapeutic agent are administered sequentially.

These and other embodiments of the invention will become apparent from the following detailed description of the preferred embodiments.

### DETAILED DESCRIPTION OF THE PREFERRED EMBODIMENTS

Traumatic brain injury (TBI) occurs when a sudden trauma causes damage to the brain. TBI affects people of all ages and manifests itself with high morbidity and mortality. The events following TBI in morbid patients result in permanent disability with lifelong financial, medical, emotional, family, and social difficulties.

A depletion of magnesium has been observed in the brain of animals and in blood of humans after brain injury. Administration of magnesium attenuated the neuro-behavioural and pathological changes in animal models of brain injury. However, two prospective clinical studies with magnesium as a neuroprotective agent in TBI patients showed variable results[1,2]. Secondary brain insults and other parameters adversely affect the clinical outcome in individuals with brain injuries. Such secondary insults may have unfavorably affected the results of the clinical studies on therapeutic efficacy of magnesium in TBI patients. Pharmacokinetic and pharmacodynamic studies in normal rats have shown that, after systemic administration, magnesium was able to enter the brain [3,4]. However, pharmacokinetic studies in humans with brain insults have shown that parenteral administration of magnesium did not cause a concomitant rise of magnesium in the CSF [5-7]. Regulation of the brain and CSF by the central nervous system may limit the blood brain barrier (BBB) permeability of peripherally administered magnesium, which could be a limiting factor in its efficacy in TBI patients. The present invention is directed to overcoming obstacles in the use of a magnesium salt to treat neurological insults.

Increasing the brain bioavailability of parenterally administered magnesium by disruption of the BBB is important to achieve the therapeutic benefits of magnesium following TBI. Increasing the brain bioavailability of magnesium using a hypertonic osmotic agent allows for a low and safe dose of magnesium to be administered to improve clinical outcome in TBI patients. Combination of magnesium and a hypertonic osmotic agent with optional pharmacological agents, like dexanabinol and progesterone, and/or physiological agents, like hyperoxia or hypothermia, provide a safe and clinically successful neuroprotective regimen for the treatment of TBI and other neurological insults.

Multiple biochemical pathways are involved in the brain degeneration process following TBI. Treatment with a single agent may result in lack of efficacy at a safe dose, or adverse effects at a therapeutic dose or upon repeated administration. A clinically successful neuroprotective therapy aims at controlling these pathways using multiple agents for a synergetic affect. In addition to magnesium, pharmacological agents [8,9] and physiological interventions, like hyperoxia and hypothermia, are being studied for the treatment of TBI. Among the pharmacological agents, dexanabinol and progesterone have been studied in clinical trials. Dexanabinol was safe but not efficacious in a phase III study. Progesterone is currently in a phase III clinical study. In accordance with the present invention, it has been found that increasing the brain bioavailability of magnesium with a hypertonic osmotic agent, like mannitol, along with an optional co-therapy using pharmacological agents and/or physiological interventions, provide an effective neuroprotective method for the treatment of TBI.

The present invention is directed to pharmaceutical compositions comprising (a) a magnesium salt and (b) a hypertonic osmotic agent, like mannitol, as well as use of the compositions in a method of treating individuals who have suffered a neurological insult. A non-limiting example of the present invention is a pharmaceutical composition comprising (a) magnesium sulfate, magnesium chloride, or a mixture thereof, and (b) mannitol together with optional excipients and/or pharmacologically acceptable carriers. In another example, the composition comprises a magnesium salt and hypertonic saline. In some embodiments of the present invention, the pharmaceutical composition comprises a magnesium salt and hypertonic osmotic agent in a weight ratio from about 1:0.1 to about 1:100. In one example, a present composition comprises magnesium sulfate and mannitol in a weight ratio from about 1:0.5 to about 1:10.

The methods, materials, and examples described herein are illustrative only and are not intended to be limiting. Materials and methods similar or equivalent to those described herein can become used in practice or testing of the invention. Other features and advantages of the invention will be apparent from the following detailed description of the preferred embodiments and the claims.

The use of the terms “a,” “an, “the,” and similar referents in the context of describing the invention, including the claims, are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The terms “treatment,” “treated,” and “treating” includes reversing, reducing, ameliorating, or arresting one or more of the symptoms, clinical signs, and underlying pathology of a condition in a manner to improve or stabilize a subject's condition.

The term “neurological insult” as used herein refers to any injury, trauma, or disease to the central nervous system, including, but not limited to, stroke, brain ischemia or ischemic episode, and traumatic brain injury (TBI).

Stroke is an acute neurologic injury occurring as a result of interrupted blood supply, resulting in an insult to the brain. Most cerebrovascular diseases present as the abrupt onset of focal neurologic deficit. The deficit may remain fixed, improve, or progressively worsen, usually leading to irreversible neuronal damage at the core of the ischemic focus, whereas neuronal dysfunction in the penumbra may be treatable and or reversible. Prolonged periods of ischemia result in frank tissue necrosis. Cerebral edema follows and progresses over the subsequent 2 to 4 days. If the region of the infarction is large, the edema may produce considerable mass effect with all of its attendant consequences.

An ischemic episode can be, but is not limited to, a global or focal cerebral episode, and is any circumstance that results in a deficient supply of blood to a tissue. Cerebral ischemic episodes result from a deficiency in the blood supply to the brain. The spinal cord, as a part of the central nervous system, is equally susceptible to ischemia resulting from diminished blood flow. An ischemic episode may be caused by hypertension, hypertensive cerebral vascular disease, rupture of aneurysm, a constriction or obstruction of a blood vessel (as occurs in the case of a thrombus or embolus), angioma, blood dyscrasias, any form of compromised cardiac function including cardiac arrest or failure, systemic hypotension, cardiac arrest, cardiogenic shock, septic shock, spinal cord trauma, head trauma, seizure, bleeding from a tumor, or other blood loss. “Focal ischemia,” as used herein in reference to the central nervous system, moans a condition that results from the blockage of a single artery that supplying blood to the brain or spinal cord, resulting in the death of all cellular elements (pan-necrosis) in the territory supplied by that artery. “Global ischemia,” as used herein in reference to the central nervous system, means a condition that results from general diminution of blood flow to the entire brain, forebrain, or spinal cord, which causes the death of neurons in selectively vulnerable regions throughout these tissues. The pathology in each of these cases is quite different, as are the clinical correlates. Models of focal ischemia apply to patients with focal cerebral infarction, while models of global ischemia are analogous to cardiac arrest, and other causes of systemic hypotension.

Traumatic brain injury (TBI) occurs when the brain undergoes sudden trauma and injury. There are two types of TBI: (a) a closed head injury where an object hits the head violently; and (b) a penetrating head injury where an object penetrates through the skull, thereby damaging the brain tissue. Traumatic brain injury affects people of all ages and manifests itself with high morbidity and mortality. In all injuries, the aftermath of TBI results in death or permanent disability with lifelong financial, medical, emotional, social, and family difficulties and implications.

The pathophysiology of TBI occurs in a series of three main phases. These phases stimulate a series of damaging and irreversible neurochemical cascades that cause brain cell death. During the primary phase, there is localized neuronal death at the injury site as a direct consequence of the injury. This is followed by the irreversible secondary phase, which is set in motion within minutes, hours, or days after the initial injury. During the secondary phase, ischemia-induced hypoxia occurs in the brain, which further increases intracranial pressure and decreases cerebral perfusion pressure or regional cerebral blood flow due to vasospasm and brain herniation. Ischemia is the foremost cause of high early mortalities following TBI.

Brain edema of cellular nature is frequently observed which results in an increase of intracranial pressure (ICP) for more than 5 days [10]. Increased ICP causes structural damage and herniation, reduces cerebral perfusion pressure (CPP) and cerebral blood flow leading to further exacerbation of ischemia. In TBI patients, an ICP greater than 20 mmHg has been shown to contribute to neurological deterioration and mortality [11].

Onset of the tertiary phase is unpredictable. During this phase, there is neuronal death in the cortical and subcortical areas of the brain distal from the injured site. The tertiary phase is responsible for a significant amount of the irreversible tissue damage after TBI and substantially contributes to morbidity and mortality. This phase however is amenable to medical intervention.

A variety of mechanisms, at different time points, have been shown to contribute to the tertiary injury process in the brain. For instance, there may be excitotoxicity with excessive release of glutamate. An excess of glutamate causes an overstimulation of N-methyl-D-aspartate (NMDA) receptors that control ionic channels. Glutamate excess may lead to edema caused by the entrance of excessive amounts of sodium and calcium along with water into the cells. Ischemia due to increased ICP in the primary phase also adversely affects the tertiary phase. Generation of oxygen free radicals and mitochondrial dysfunctions are observed which lead to calcium accumulation, depletion of adenosine triphosphate (ATP), generation of reactive oxygen species, and apoptosis [12,13]. Together, these result in neuro-generation. The cascade of events then would be almost irreversible and result in the degeneration of neurons and, ultimately, cell death. As such, the NMDA receptor has become a target for the development of neuroprotective agents.

Magnesium has an important role in homeostatic regulation of the pathways involved in the delayed third phase of brain injury. During normal physiological processes, magnesium is a non-competitive inhibitor of the NMDA receptors, and thus regulates calcium influx. In the cascade of events following TBI, there is a depletion of magnesium, resulting in the loss of its homeostatic control over the NMDA receptors. This leads to a massive influx of calcium and, consequently, to neuronal degeneration and cell death.

Magnesium also may reduce oxidative stress following TBI. Magnesium deficiency was associated with increased oxidative stress in rats through a reduction in plasma antioxidants and increased lipid peroxidation possibly due to increased susceptibility of body organs to free radical injury [14]. Administration of magnesium to dogs during coronary occlusion attenuated the increase of free radicals during reperfusion [15]. These findings were confirmed in TBI patients, wherein administration of magnesium sulfate reduced oxidative stress following TBI in humans [16].

In patients with subarachnoid hemorrhage undergoing temporary cerebral artery occlusion for clipping of cerebral aneurysm, treatment with magnesium sulfate dilated the leptomeningeal arteries and enhanced collateral blood flow and tissue oxygenation [17].

Molecular mechanisms have been studied on the efficacy of magnesium in attenuating the neurological damage in TBI. The tumor-suppressor gene p53 is a regulator of neuronal apoptosis. Up-regulation of p53 mRNA was observed in the cortex, thalamus and hippocampus following brain injury in rats [18]. Treatment with magnesium reduced the upregulation of p53 gene and apoptosis in rats with brain injury [19].

Water homeostasis is critical for optimal neuronal function and any alteration of intracellular and extracellular water content will disrupt ionic homeostasis and electrical conduction [20]. Aquaporin-4, a membrane protein found in the brain astrocytes of mammals, has an important role in the homeostasis of water. Aquaporin-4 is upregulated in brain injury, leads to an increase in the brain water content and results in brain edema. In rats with brain injury, magnesium down-regulated aquaporin-4 channels [21], and thereby attenuated brain edema [22].

The generation of oxygen free radicals has been observed with TBI. Mitochondrial dysfunctions have been observed following TBI. During the secondary phase of damage to the brain following TBI, acute inflammatory response is initiated by the infiltration, accumulation, and activation of polymorphonuclear leucocytes at the injury site. These leucocytes increase post traumatic brain swelling, size of contusions, and abnormal lesions. Activated leucocytes produce pro-inflammatory cytokines, like TNF-α IL-1, IL-6, leukotrienes, complement, integrin, and platelet-activating factor (PAF). These cytokines have a deleterious role in the pathological cascade by altering vascular permeability, and inducing brain edema, leading further to the influx of inflammatory cells and oxygen free radical production. During the tertiary injury process following TBI, several imbalances in the biochemical homeostasis pathways and factors have been observed to contribute to the cascade of events leading to the injury. It appears that a single pathway or factor is not responsible for causing such a massive damage to the brain.

Magnesium is theorized to have an important role in the pathophysiological events following TBI. A disruption of magnesium homeostasis has been observed after TBI and normalizing magnesium levels has resulted in improved neurological recoveries. Ionized free magnesium concentration following TBI is a prognostic indicator of long-term neurobehavioral and motor outcome in rats. A decline in intracellular free magnesium concentration following TBI may represent an early critical factor for irreversible brain damage, and early measurement of ionized magnesium could be a useful clinical predictor of the late outcome after head injury.

A disruption of magnesium homeostasis has been observed after brain injury, and normalizing magnesium levels has resulted in improved neurological recoveries. A decline in free extracellular and intracellular magnesium concentrations has been observed after TBI. The decrease in free magnesium concentration following TBI has been correlated with the neurological outcome and behavioral deficits [23] following graded traumatic axonol brain injury, fluid percussion injury, and impact-acceleration-induced injury in rats. A significant and linear correlation has been observed between decreased ionized magnesium concentrations at 24 hours following fluid percussion injury and 1 and 2 weeks [24] thereafter on neuromotor deficits in rats. In a rat model of fluid percussion injury, magnesium concentration declined significantly within hours after injury which persisted for 5 days before recovering to pre-injury levels. It has been suggested that the tertiary process of the damaging cascade is continuing during declined magnesium levels. This provides along window of opportunity of about 5 days for a delayed therapeutic intervention or a continuous infusion to normalize magnesium homeostasis.

Several studies in rats have shown that treatment with magnesium following brain injury had neuroprotective effects on motor and behavioral outcome [25-28] in a dose-dependent manner [29-30]. Cortical damage was attenuated after treatment with magnesium in rats [31]. Magnesium reversed persistent motor and cognitive deficits with reduction of post-traumatic stress and anxiety following brain injury in rats. Magnesium has shown a neuroprotective role and improved motor outcome and behavioral parameters following severe diffuse traumatic axonal brain injury when administered up to 24 hours following diffuse traumatic axomal brain injury in rats [32, 33] and electrolytic lesions of the sensorimotor cortex. Magnesium therapy in a dose-dependent manner effectively facilitated recovery of sensorimotor functions and reduced working memory deficits and cognitive functions following cortical lesions in rats.

Deprivation of magnesium during experimental brain trauma exacerbated neurological deficits, whereas post-traumatic supplementation with magnesium benefited the neurological outcome in an electrolytic lesion model of cortical injury in rats. In a rat model of closed head trauma, administration of magnesium after one hour attenuated brain edema formation and improved neurological outcome. Protective effects of magnesium against blood-brain barrier breakdown in diffuse TBI models in rats also have been observed.

Despite reports of such benefits, magnesium therapy following TBI may not always improve the mortality and morbidity of the subjects. In many severe TBIs, a subdural hemotoma that develops subsequent to the primary event causing the injury is observed. In a rat model of impact acceleration diffuse brain trauma, which frequently produces extensive subdural hematoma, administration of magnesium produced significant improvements in motor activities in those rats which showed no subdural hematoma during postmortem examination. In those rats with subdural hematoma observed during postmortem examination, no improvements in motor deficits were observed upon administration with magnesium. In the brain, free magnesium concentration in the magnesium treated/hematoma group demonstrated a biphasic decline, i.e., an initial immediate decline, then recovery of brain magnesium levels subsequent to magnesium treatment, and then a significant subsequent decline of brain magnesium concentration. The subsequent decline in brain magnesium concentration is not observed in the magnesium treated/no hematoma group of rats. Development of subdural hematoma following TBI results in a decline of brain magnesium concentration, even after magnesium treatment.

Examples of central nervous system disorders treatable by the present invention include, but are not limited to, increased intracranial pressure, brain edema due to head injury, intoxications, hepatic failure, space-occupying cerebral lesions, meningitis, Reye's syndrome, cerebral malaria, and brain tumors. In addition, a present composition can be used for treatment of patients suffering from disorders including, but not limited to, birth asphyxia, perinatal asphyxia, asphyxiated neonates and infants, rebound phenomenon in the treatment of raised intracranial pressure, hyperglycemic crisis and its complications, diabetic ketoacidosis, acute stroke, ischemic stroke, cerebral hemorrhage, focal ischemia, subarachnoid hemorrhage, drug induced hypomagnesaemia, neurological complications of chemotherapeutic agents, preeclampsia, epileptic episodes, prolongation of opiate and non-opiate analgesia, affective disorders, post-traumatic depression/anxiety, neuropsychiatric disorders, headaches and migraines, and neuroprotection of the adult and neonatal brain.

A present pharmaceutical composition comprises (a) a magnesium salt and (b) a hypertonic osmotic agent, and typically an excipient and/or pharmaceutically acceptable carrier. One embodiment of the present invention is a pharmaceutical composition consisting essentially of (a) a magnesium salt, such as magnesium sulfate, magnesiumchloride, or both and (b) mannitol.

The term “consisting essentially of” is a transitional phrase that, when it precedes a list of components or a series of steps, indicates that while the listed components or steps are necessary to the claimed invention, any unlisted components or steps that do not materially affect the basic and novel properties of the invention are contemplated as within the scope of the claimed invention.

Examples of magnesium salts include, but are not limited to, magnesium chloride and magnesium sulfate. In preclinical studies, both have been studied. In a comparative study, no differences were observed between the benefits of treatment with either magnesium chloride and magnesium sulfate in a rat model of diffuse axonal injury on motor deficits. Magnesium sulfate has been studied in clinical trials of TBI and stroke.

Ionized magnesium is the physiologically active form that can enter the brain, and its levels are affected by the total magnesium concentration in the CSF. CSF magnesium concentration is used as a surrogate marker of brain magnesium concentration [6]. In a comparative analysis of serum and CSF magnesium concentrations in TBI patients with a mean Glasgow Coma Scale (GCS) score of 8.7, serum ionized magnesium concentration correlated with the GCS scores [36]. In another study, elevated magnesium levels were observed in the ventricular CSF of TBI patients with a mean GCS score of 5.6 [37]. In humans with graded TBI with GCS scores of 4-6 (extensive penetrating injury) and 13-15 (mild, closed injury), a time-dependent increase of plasma ionized magnesium was observed for 7 days [16]. This study observed a persistent production of reactive oxygen species malondialdehyde and a delayed decrease of the anti-oxidant superoxide dismutase, suggesting increased anti-oxidant utilization. The study showed a correlation between the decline in plasma ionized magnesium concentration and the development of oxidative stress in TBI.

The safety and tolerability of magnesium has been studied in adult and pediatric patients with TBI. In the Turin Lidomag Pilot Study, a high dose of magnesium and low dose of lidocaine were administered for 3 days to 32 adult patients with immediate and severe TBI and having a Glasgow Coma Scale (GCS) score of 3-8 [34]. Several studies have shown that the 3 day window is a critical period to provide maximal neuroprotection. Magnesium was administered intravenously at an initial dose of 70 mg/kg followed by a maintenance dose of 15 mg/kg/hour. Lidocaine was administered at an initial dose of 1.5 mg/kg administered intravenously, followed by a maintenance dose of 1 mg/kg/hour administered intravenously. The patients were monitored for 6 months. The study showed that a combination of magnesium and lidocaine was safe and well tolerated with a reduced mortality.

The safety of magnesium has been studied in pediatric patients suffering from TBI [35]. Six pediatric patients ranging from 3.4 to 15.4 years of age and GCS score of 3-11 were recruited, 4 of which were placed on magnesium dosing within 17-56 hours of injury. Two patients served as controls and were administered with normal saline. Magnesium was administered at an initial dose of 50 mg/kg and up to 4 gram maximum, administered intravenously over 30 minutes in normal saline at a concentration of 50 mg/mL. This was followed by a maintenance dose of 8.3 mg/kg/hour, IV, for 24 hours. A long term follow up with neuropsychological testing and brain magnetic resonance imaging (MRI) was done at 3 months after injury. No adverse hemodynamic effects were observed in these pediatric patients with TBI.

In contrast to the preclinical studies, clinical studies with magnesium treatment following TBI has failed to show consistent beneficial effects. Two prospective clinical studies have reported variable effects of magnesium in TBI patients.

In a clinical trial with 499 patients [1], administration of magnesium within 8 hours of moderate to severe TBI for 5 days did not show any beneficial effects on the composite primary outcome based on survival, seizures, measures of functional status, and a comprehensive battery of neuropsychological tests which are sensitive to the integrity of the brain conducted at 6 months post-injury. Magnesium was administered by continuous infusion to achieve consistent levels and dosing was adjusted to achieve magnesium serum concentration ranging from 1-1.85 to 1.25-2.5 mM/L. At the lower dose of magnesium, the composite score was worse than the placebo treated patients. Significantly higher mortality was observed in patients treated with high dose of magnesium. It is possible that despite achieving the desired concentrations of magnesium in the serum, the brain concentrations were not increased to warrant a beneficial outcome.

In another clinical trial consisting of 30 patients with acute brain injury secondary to subarachnoid hemorrhage, TBI, primary intra-cerebral hemorrhage, subdural hematoma, brain tumor, CNS infection, and ischemic stroke, the patients were infused with magnesium on average of 5 days post injury (range 1-16 days) for 24 hours. The magnesium dose was adjusted to achieve serum concentration of 2.1-2.5 mM/L. At the end of the infusion, serum magnesium concentrations doubled from baseline, but the CSF total magnesium increased by 15% and the ionized magnesium increased by 11% relative to baseline values. CSF magnesium concentration is used as a surrogate marker of brain bioavailability. Serum hypermagnesia thus produced only marginal increases in CSF total and ionized magnesium concentrations. These two clinical studies may not be correlated due to the time delay in the onset of magnesium administration.

In another clinical study in 60 patients with closed head TBI and Glasgow Coma Score of 5-8, magnesium was administered within 12 hours of injury as an initial dose by both intravenous (4 g over 5-10 minutes) and intramuscular (10 g) routes, followed by a maintenance dose of 5 g administered intramuscularly every 4 hours for 24 hours. At the end of three months, favorable outcome including reduced mortality and intra-operative brain swelling was observed in 73% (22/30) of the patients administered with magnesium and 40% (12/30) of the patients in the control group.

In a retrospective analysis of a prospective clinical trial, magnesium therapy failed to provide a favorable outcome at six months. Patients with a lower initial serum magnesium concentration (<1.3 mEq/L) who were supplemented with magnesium had a worse outcome at 6 months than those patients in whom the serum magnesium levels were not supplemented within 24 hours.

The pathophysiology of stroke and TBI are similar, and magnesium therapy has been studied in several clinical trials of stroke and subarachnoid hemorrhage. The safety and tolerability of magnesium was studied in 60 stroke patients in whom magnesium was administered intravenously within 12 hours of the diagnosis at a dose of 8 mmol over 15 minutes followed by 65 mmol over 24 hours or 2.7 mM/h. Serum magnesium level rose from 0.76 mM/L to 1.42 mM/L over 24 hours and remained significantly higher than in the saline placebo group at 48 hours. No differences in blood pressure or adverse events between the magnesium- and placebo-treated patients were observed. It was concluded that magnesium is a safe and feasible potential therapy in acute stroke.

In another placebo-controlled, double-blind clinical study in patients with acute stroke, magnesium was administered within 24 hours stroke onset intravenously at a loading dose of 16 mM and then infusion at 6 mM/hour for 5 days. After one month of follow up, several outcome scales (Orgogozo, Mathew, Rankin) indicated that magnesium treatment had a significant positive effect on patient outcome.

The dosing regimen for a prospective clinical trial (IMAGES) was studied in another clinical trial in which magnesium was administered intravenously to 25 patients within 24 hours of the onset of stroke at a loading dose of 8, 12 or 16 mM, followed by a 65 mM infusion over 24 hours. This dose optimization pharmacokinetic study showed that magnesium could be given in a regimen to stroke patients which provided magnesium levels that produced neuroprotection in rat models of stroke. There were no obvious effects of magnesium on heart rate, blood pressure, or blood glucose. The 16 mmol loading infusion achieved target serum concentrations (1.49 mmol/L) most rapidly. Survival curve analysis found a trend in favor of magnesium, though no significant differences in outcome measures were observed in magnesium and placebo-treated groups.

IMAGES was an international, multiple-center, double-blind, placebo-controlled stroke trial that revealed the efficacy of intravenous magnesium. In this trial, 2,589 patients were randomly assigned (efficacy dataset n=2386). Most of the patients received magnesium sulfate treatment (16 mmol of bolus injection and 65 mmol of continuous infusion over 24 hrs) beyond 3 hrs (up to 12 hrs) of symptom onset. Most of the findings of this study were disappointing because the primary outcome was not improved by the magnesium. Furthermore, the mortality was slightly higher in the magnesium treatment group, and the secondary outcomes did not show a treatment effect. Further subgroup analysis of the IMAGES trial data revealed that magnesium treatment improved the chances of good functional outcome in patients with clinical lacunar syndrome.

In the FAST-MAG Pilot Trial, 20 patients were enrolled. Magnesium sulfate infusion (10 mmol of bolus injection and 64 mmol of continuous infusion over 24 hrs) began a median of 100 minutes after symptom onset (range 24-703 minutes), and 70% received the study agent within 2 hrs of onset. The interval from paramedic arrival on the scene to study agent start was as follows: field-initiated, 26 minutes (range 15-64) vs. in-hospital initiated (historic controls), 139 minutes. Paramedics rated patient status on hospital arrival as improved in 20% of cases, worsened in 5%, and unchanged in 75%. Median National Institutes of Health Stroke Scale (NIHSS) on hospital arrival was 11 in all patients and 16 in patients unchanged since the initiation of field treatment. Good functional outcome at 3 months occurred in 60% of patients. The reason for the discrepancy in findings between the IMAGES and FAST-MAG clinical studies is unclear.

The effect of magnesium was studied in 283 patients with aneurysmal subarachnoid hemorrhage in the placebo-controlled MASH clinical trial. It was observed that magnesium reduced delayed cerebral ischemia and the subsequent poor outcome, but the results were not definitive.

The characteristics of a neuroprotective agent include (a) entry into the central nervous system; (b) presence in the central nervous system at a concentration known to be neuroprotective; and (c) presence of an adequate concentration in the brain during an interval that will improve neuronal survival. Disruption of the blood brain barrier has been generally observed shortly following experimental TBI in rats. Increased brain magnesium concentrations were observed following its intravenous administration in rats following TBI, which correlated linearly with its dose and neurological outcome. Free ionized and total magnesium concentrations vary with the severity of TBI. In pediatric patients with TBI, the total serum magnesium was decreased after mild, moderate or severe TBI and remains low for more than 24 hours, while ionized magnesium was decreased in severe TBI with a Glasgow Coma Scale of <8, but it normalizes within 24 hours. This may suggest that ionized magnesium can serve as a marker for a limited period of time and the presence of a mechanism working towards normalizing it. Only the ionized, physiologically active form of magnesium can enter the brain, but the total magnesium represents the concomitant changes in ionized magnesium.

In an analysis of serum and CSF magnesium and calcium concentrations in patients with severe head injury, it was observed that the serum ionized magnesium concentration affects the neurologic state through the CSF ionized magnesium concentration. However, in patients with mild or moderate head injury, the ionized magnesium concentration also may be associated with the degree of neurologic deficit based on the ionized calcium level. The CSF and serum ionized magnesium dissociation may thus result from the slow movement of ionized magnesium through the blood-brain barrier. An understanding of the brain bioavailability of magnesium is important to assess its potential as a neuroprotective agent following TBI. Inducing hypermagnesemia peripherally will likely cause an increase in magnesium concentration in the brain. It has been suggested that following TBI, magnesium may be administered with a reperfusion agent. In rat modes of ischemic stroke, beneficial effects have been observed using combined therapy with magnesium and tirilazad, an antioxidant, along with hypothermia.

The blood-brain barrier (BBB) is made up of brain microvessel endothelial cells characterized by tight intercellular junctions, minimal pinocytic activity, and the absence of fenestra. These characteristics endow these cells with the ability to restrict passage of most small, polar blood-borne molecules (e.g., neurotransmitter catecholamines, small peptides) and macromolecules (e.g., proteins) from the cerebrovascular circulation to the brain. The blood-brain barrier contains highly active enzyme systems as well, which further enhance the already very effective protective function. It is recognized that transport of molecules to the brain is not determined solely by molecular size, but by the permeabilities governed by specific chemical characteristics of the permeating substance. Thus, besides molecular size and lipophilicity, the affinity of the substances to various blood proteins, specific enzymes in the blood, or the blood-brain barrier considerably influence the amount of the drug reaching the brain.

Preclinical studies using 25% mannitol to disrupt the BBB have been studied in rats, rabbits, dogs, and baboons. In rats, the BBB was opened by intracarotid infusion of 25% mannitol at a rate of 0.25 mL/kg/sec for 30 seconds. Mannitol was administered in 0.9% saline, filtered, and warmed to 37° C. before infusion. In dogs, the BBB was disrupted by intracarotid infusion of 25% mannitol at a rate of 1.5 mL/sec for 30 seconds. In baboons, 25% mannitol was infused for BBB disruption (BBBD).

Human clinical studies exploited the disruption of BBB by mannitol to get drugs into the brain that otherwise would not enter brains with intact BBB. The cognitive functions were found to be preserved following BBB disruption for cancer chemotherapy in humans. The following techniques have been used in clinical trials in which mannitol-induced BBB disruption was used to deliver anti-cancer drugs to the brain.

Osmotic blood-brain barrier disruption was performed under general anesthesia via a transfemoral catheter placed into either the distal cervical internal carotid artery or in a vertebral artery at the level of the sixth cervo-vertebral body. The blood-brain barrier then was disrupted by the intraarterial injection of 25% mannitol. The flow rate of mannitol varies from 5 to 12 mL per second, and the duration of the injection was 30 seconds. The flow rate was selected to deliver enough mannitol to disrupt the blood-brain barrier.

Selective catheterization via percutaneous transfemoral puncture of the left internal carotid artery, right internal carotid artery, and left or right vertebral artery was performed by determining rate of infusion of mannitol by iodinated contrast injection and fluoroscopy at the lowest infusion rate in which there is retrograde flow from the arterial catheter. The volume of mannitol infused is determined in mL/second×30 seconds (usually between 4 and 12 mL/s in the carotid circulation, and between 4 and 10 mL/s in the vertebral circulation). Osmotic disruption of the BBB was achieved by infusing 25% mannitol in the previously catheterized artery at the defined rate. Infuse contrast medium was used to confirm catheter position and rule out arterial injury after disruption.

BBB opening and its termination were performed with the patient under general anesthesia. A transfemoral intraarterial catheter was placed in an internal carotid artery at C1-2 or in a vertebral artery at C4-5. Warmed mannitol (25%) was administered at 4-10 mL/second into the cannulated artery for 30 seconds (the precise flow rate was determined by fluoroscopy).

The extent of BBB disruption was confirmed by contrast CT immediately. Iodinated contrast agent is administered 5 minutes after the disruption for that purpose. Seizures (generally focal) occur during approximately 25% of BBB disruption treatments, patients are pre-medicated with an anticonvulsant. To prevent bradycardia, atropine is administered intravenously immediately prior to BBB disruption.

Perhaps the most serious side/adverse effect of mannitol is fluid and electrolyte imbalance. Rapid administration of large doses may lead to accumulation of mannitol, overexpansion of extracellular fluid, dilutional hyponatremia and occasional hyperkalemia, and circulatory overload, especially in patients with acute or chronic renal failure. Inadequate hydration or hypovolemia may be obscured by the diuresis produced by mannitol, which may lead to tissue dehydration, promotion of oliguria, and intensification of pre-existing hemoconcentration. Extravasation of mannitol may result in edema and skin necrosis.

Mannitol induced renal toxicity has been observed in humans when the serum mannitol concentration exceeds (1000 mg/dL, corresponding to an osmolal gap of >55 mosm/kg of water), leading to acute reduction of GFR and renal failure. Acute renal failure and its duration is when serum creatinine is >2 mg/dL.

Afferent arteriolar vasoconstriction may be the most likely cause of mannitol-induced acute renal failure. Mannitol half life in humans with normal renal function is 1.2 hours and it increases to 36 hours in uremia patients. The renal failure responds to withdrawal of mannitol and to hemodialysis, with resumption of diuresis and complete recovery of renal functions.

Mannitol concentration can be calculated by measuring the serum osmolal gap. Osmole is the molecular weight of a solute, in grams, divided by the number of ions or particles into which it dissociates in solution. It is also a unit of osmotic pressure equivalent to the amount or solute substances that dissociates in solution to form one mole (Avogadro's number) of particles (molecules and ions). Osmolality is a measure of the osmoles of solute per kilogram of solvent.

Calculated serum osmolality=2×Na(mEq/1)+glucose(mg/dL)/18+BUN(mg/dL)/2.8.

Serum osmolal gap(Osm gap)=Measured−Calculated serum osmolality.

Serum mannitol(mg/dL)=Osmolal gap×18.2(Mannitol molecular weight:182).

Acute renal failure occurs with mannitol concentration at >1000 mg/dL which corresponds to an osmolal gap of >55 mosm/kg of water.

The osmolal gap must be monitored during mannitol therapy, especially in patients with renal dysfunction. Osmolal gap aids in the diagnosis of mannitol-induced acute renal failure. Patients with renal dysfunction may show mannitol accumulation from lower dose infusion, compared with patients whose renal function is normal. Mannitol is reported to be metabolically inert and accumulates when the rate of infusion exceeds the rate of urinary excretion.

Acute renal failure with mortality (2/4) has been reported following massive mannitol infusion to 4 male adults between the ages of 20 an 42 years. 1.172+0.439 kg (Mean+SD) of mannitol was administered over 58+28 hours. The onset of acute renal failure was detected 48±22 h after infusion. The deaths occurred from endocranial hypertension.

In addition to mannitol, other hypertonic osmotic agents can be used in conjunction with a magnesium salt. Hypertonic osmotic agents useful in the present composition include mannitol, hypertonic saline (2.5-10% NaCl), hypertonic saline with dextran, hypertonic saline with hetastarch, dextran (5 to 50%), inulin, hetastarch (ethoxylated amylopectin), pentastarch (hydroxyethyl starch), urea, glycerol, arabinose, sucrose, lactamide, and mixtures thereof.

Typically, the composition contains about 5% to about 25%, and preferably about 8% to about 20%, by weight, of the magnesium salt. To achieve the full advantage of the present invention, the composition contains about 10% to about 20%, by weight, of the magnesium salt.

In addition, a present composition contains about 3% to about 25%, and preferably about 5% to about 25%, by weight, of the hypertonic osmotic agent. To achieve the full advantage of the present invention, the hypertonic osmotic agent is present in an amount of about 5% to about 20%, by weight of the composition.

The present invention also is directed to a method of treating an individual who has suffered a neurological insult comprising administering to the individual a therapeutically effective amount of a pharmaceutical composition comprising a magnesium salt and a hypertonic osmotic agent, such as mannitol. In another embodiment, the method comprises administering therapeutically effective amounts of a magnesium salt and a hypertonic osmotic agent from separate compositions.

The term “therapeutically effective” refers to an amount sufficient to effect treatment, when administered to an individual in need of such treatment. A therapeutically effective amount varies depending on the subject being treated (e.g., age, weight), the severity of the disease state, and the manner of administration, and can be determined routinely by one of ordinary skill in the art. A therapeutically effective amount also can be one in which a toxic or detrimental effect of the treatment is outweighed by a therapeutically beneficial effect.

The term “administering” or “administration” refers to the delivery of a drug to an individual. The treatment regimen is carried out in terms of administration mode, timing of the administration, and dosage, such that the functional recovery of the patient from the adverse consequences of the ischemic events or central nervous system injury is improved; i.e., the patient's motor skills (e.g., posture, balance, grasp, or gait), cognitive skills, speech, and/or sensory perception (including visual ability, taste, olfaction, and proprioception) improve as a result of administration of a composition of the invention. According to the present method, the magnesium salt and hypertonic osmotic agent can be concurrently administered from separate compositions.

“Concurrent administration,” “administered in combination,” “simultaneous administration” and similar phrases mean that two or more agents are administered concurrently to the subject being treated. By “concurrently,” it is meant that each agent is administered simultaneously or sequentially in any order at different points in time. However, if not administered simultaneously, they are, in one aspect, administered sufficiently closely in time so as to provide the desired treatment effect of the combination of agents. Suitable dosing intervals and dosing order of the agents will be readily apparent to those skilled in the art. It also is contemplated that two or more agents are administered from separate compositions, and in one aspect, one composition is administered prior to administration of the other composition.

Routes of administration are well known to skilled pharmacologists and physicians and include intraperitoneal, intramuscular, subcutaneous, and intravenous administration. Additional routes include intracranial (e.g., intracisternal or intraventricular), intraorbital, ophthalmic, intracapsular, intraspinai intraperitoneal, transmucosal, topical, subcutaneous, and oral administration.

Typically, the magnesium salt is administered intravenously in an amount of about 0.1 to about 10 g (as magnesium sulfate) in a 10% to 20% solution, by weight, or about 0.5 to about 8 grams per dose, preferably about 1 to about 4 grams per dose. The hypertonic osmotic agent is administered in an amount of about 1 to about 1000 g, intravenously in 5%, 10%, 15%, or 20% solution, or about 5 to about 500 g per dose, or about 5 to about 500 g per dose, or preferably about 20 to about 100 grams per dose.

The present invention also contemplates use of the pharmaceutical composition co-administered with a pharmaceutically acceptable carrier.

The term “pharmaceutically acceptable” as used herein refers to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A present composition can contain additional therapeutic agents useful in the treatment of a neurological insult. Such additional agents also can be administered from a separate composition in a method of the present invention. The additional therapeutic agents are different from a magnesium salt and hypertonic osmotic agent. An additional therapeutic agent can be pharmacological or physiological. For example, a pharmacologic agent can be the antioxidant tirilazad. A combination therapy with magnesium and the antioxidant tirilazad significantly improved neurologic function and reduced infarct volume in rats with cerebral ischemia [38]. Further, a combination of magnesium and vitamin B₂ riboflavin significantly improved functional recovery in rats subjected to cortical contusion injury [39].

Additional pharmacological agents include statins, progestereone, erythropoietin, minocycline, Toll-like receptor agonists, dexanabinol, thyrotropin releasing hormone analogs, and cyclosporin-A, which were evaluated pre-clinically for the treatment of brain injury [8, 9]. Of these, dexanabinol and progesterone were studied clinically for the treatment of TBI [40-42].

A physiological agent can be hypothermia or hyperoxia. A combination therapy with magnesium and hypothermia reduced neuronal death [43] and infarct volume [44] in rats with cerebral ischemia. A synergistic reduction in infarct volume was observed with the combination of magnesium, tirilazad, and hypothermia in rats with cerebral ischemia [45] in a post-injury time-dependant manner [46]. Treatment of TBI patients with normobaric hyperoxia has shown variable results.

The magnesium salt, hypertonic osmotic agent, and additional therapeutic agent can be administered simultaneously or sequentially to achieve the desired effect. In addition, the magnesium salt, hypertonic osmotic agent, and additional therapeutic agent can be administered from a single composition or two or three separate compositions. The magnesium salt, hypertonic osmotic agent, and additional therapeutic agent can be administered together as a single-unit dose or separately as multi-unit doses. One or more doses of each agent can be administered.

The additional therapeutic agent is administered in an amount to provide its desired therapeutic effect. The effective dosage range for each additional therapeutic agent is known in the art, and the additional therapeutic agent is administered to an individual in need thereof within such established ranges.

One example of a composition of the present invention is prepared using the following ingredients:

### D-mannitol, Fisher-Scientific Certified A.C.S., FW: 182.17, CAS 69-65-8; and

magnesium sulfate (MgSO₄), Aldrich Chemical Company, FW: 120.37, CAS 7487-88-9.

Composition Preparation:

### Example 1

Dissolve 100 g (gram) of D-mannitol in 500 ml (milliliter) of sterile water. Stir and heat at 35 to 40° C. for 15 minutes to provide a clear solution. Then, add 20 grams of magnesium sulfate to the clear mannitol solution. Stir and heat at 55 to 60° C. for 30 minutes to provide a clear solution. The pH was about 8.72, and hydrochloric acid was added to adjust the pH to about 7.4. The composition was filtered, autoclaved, and stored.

### Example 2

% Mannitol Injection, volume 50 ml, containing 2 g magnesium sulfate was prepared. The composition was administered intravenously 4 times a day (6 hourly) over 30-60 minutes. Store at room temperature 15°-30° C. (59°-86° F.).

### Example 3

Mannitol 10 g and 2 g of magnesium sulfate per 50 ml. Mannitol (10 g) was prepared in 45 ml of water. Separately magnesium sulfate (2 g) was prepared in 5 ml of water. The two solutions were mixed until a clear solution resulted (pH 6.3 (4.5 to 7.0). Sodium bicarbonate and/or hydrochloric acid can be added for pH adjustment.

The following example illustrates a method of the present invention:

### Material and Methods

Inclusion criteria: (a) age 18 years or older; (b) gender male or female; (c) mechanically ventilated unstable condition for >2 hours prior to study; (d) serum osmolality between 280-320 mmol/kg; and (e) patient with infarct between 6 to 24 hours of symptoms.

Exclusion criteria: (a) imminent cranial or extra cranial surgery; (b) leakage or drainage of CSF; (c) unstable respiratory or hemodynamic condition; (d) renal failure; (c) anemia; (f) prior use of mannitol, hyperosmotic agent, diuretics or steroids; (g) pulmonary edema; (h) acute left ventricular failure, congestive heart failure; (i) hypersensitivity to the drug; (j) patients with cerebral hemorrhage, subdural hemorrhage, epidural hemorrhage or subarachnoid hemorrhage; (k) pregnancy; (l) cerebral degenerative demyelinating diseases; (m) brain tumor; (n) previous stroke; and (o) metabolic disorder excluding diabetes mellitus.

### Criteria for Evaluation

Following tests are carried out three times, each: upon admission (baseline—before treatment); five days after start of treatment; fifteen days after start of treatment; thirty days after start or treatment; and ninety days after start of treatment (in some studies).

Tests performed: Glasgow outcomes scales (GOS); Barthel Index (BI); Modified Rankin Score (MRS); Body temperature every 6 hours; Complete hematological examination; X-ray chest; Fundus examination by ophthalmologist; lumbar puncture and CSF examination; CSF examination PCR for tuberculosis or other infections; CSF and blood magnesium (total and ionized level); CSF and blood calcium (total and ionized level); CT Scan/MRI; EEG; and blood electrolyte estimation.

Glasgow Outcome Scale:

### DEFINITION OF TERMS

### 1. Dead

2. Vegetative State—Unable to interact with environment; unresponsive.
3. Severe Disability—Able to follow commands/unable to live independently.
4. Moderate Disability—Able to live independently; unable to return to work or school.
5. Good Recovery—Able to return to work or school.

Glasgow Coma Scale:

| | | |
|---|---|---|
| Eye Response: | 4. | Spontaneous eye movement |
| | 3. | To Verbal commands |
| | 2. | To Pain stimuli |
| | 1. | No response |
| Best Verbal Response: | 5. | Oriented |
| | 4. | Disoriented |
| | 3. | Inappropriate Speech |
| | 2. | Incomprehensible words |
| | 1. | No response |
| Best Motor Response: | 6. | Obeys command |
| | 5. | Withdrawal to pain stimulus |
| | 4. | Localization to pain |
| | 3. | Decorticate rigidity |
| | 2. | Decerebrate rigidity |
| | 1. | No Response |

Barthel Index (BI):

| | | With | |
|---|---|---|---|
| | | help | Independent |
| 1. | Feeding (if food needs to be cut up help) | 5 | 10 |
| 2. | Moving from wheel chair to bed & return | 5-10 | 15 |
| | (includes sitting up in bed) | | |
| 3. | Personal toilet (wash face, comb, hair, | 0 | 5 |
| | shave clean, teeth) | | |
| 4. | Getting on & off toilet (handling clothes, | 5 | 10 |
| | wipe, flush) | | |
| 5. | Bathing self | 0 | 5 |
| 6. | Waling on level surface (or if unable to | 0 | 5 |
| | walk, propel wheel chair) Score only if | | |
| | unable to walk | | |
| 7. | Ascend & descend stairs | 5 | 10 |
| 8. | Dressing (includes tying shoes, fastening | 5 | 10 |
| | fasteners) | | |
| 9. | Controlling bowels | 5 | 10 |
| 10. | Controlling bladder | 5 | 10 |

A patient scoring 100 BI is continent, feeds himself, dresses himself, gets up out of bed and chairs, bathes himself, walks at least a block, and can ascend descend stairs. This does not mean that he is able to live alone. The patient may not be able to cook, keep house and meet the public, but he is able to function without attendant care.

Modified Rankin Scale:

| Score | Description |
|---|---|
| 0 | No symptoms at all. |
| 1 | No significant disability despite symptoms; able to |
| | carry out all usual duties and activities. |
| 2 | Slight disability; unable to carry out all previous |
| | activities, but able to look after own affairs without |
| | assistance. |
| 3 | Moderate disability; requiring some help, but able to |
| | walk without assistance. |
| 4 | Moderately severe disability; unable to walk without |
| | assistance and unable to attend to own bodily needs |
| | without assistance. |
| 5 | Severe disability; bedridden, incontinent and requiring |
| | constant nursing care and attention. |
| 6 | Dead |
| Total | 0-6 |

Functional Independence Measure TM and Functional Assessment Measure

Brain Injury Scale:

| | |
|---|---|
| 7 | Complete Independence (timely, safely) |
| 6 | Modified Independence (extra time, devices) |
| 5 | Supervision (cuing, coaxing, prompting) |
| 4 | Minimal Assist (performs 75% or more of task) |
| 3 | Moderate Assist (performs 50%-74% of task) |
| 2 | Maximal Assist (performs 25% to 49% of task) |
| 1 | Total Assist (performs less than 25% of task) |

Self Care Items:

feeding, grooming, bathing, dressing upper body, dressing lower body, toileting, swallowing

Sphincter Control:

bladder management, bowel management

Mobility Items: (Type of Transfer) bed, chair, wheelchair, toilet, tub or shower, car transfer

Locomotion:

walking/wheelchair (circle), stairs, community access

Communication Items:

comprehension-audio/visual (circle)

expression-verbal, non-verbal (circle)

reading, writing

speech intelligibility

Psychosocial Adjustment:

social interaction, emotional status, adjustment to limitations, employability

cognitive function:

problem solving, memory, orientation, attention, safety judgment

Safety—safety of subjects was maintained at all times. Adverse events and clinical laboratory data were obtained. Serious and non-serious adverse effects and clinical laboratory data were compared between groups.

Study duration—Following established baseline assessment of stroke severity, following treatment of magnesium sulfate, subjects participate in the study for up to 30 days.

### Mode of Study (Four Groups)

Group 1. Magnesium sulfate 16 gm a day (4 vials each of 1 gm of magnesium sulfate mixed with 100 ml normal saline was infused 6 hourly for five days)

Group 2. Mannitol 100 ml (20%) mannitol once a day for five days

Group 3. Magnesium sulfate 8 gm a day plus mannitol (2 vials each of 1 gm of magnesium sulfate mixed with 50 ml normal saline was infused 6 hourly for five days; in addition 100 ml of 20% mannitol once a day with first dose of magnesium sulfate was administered)

Group 4. Control group—No magnesium or mannitol infused

The following scales were used for evaluation at day 1, day 5, day 15, and day 30:

| | |
|---|---|
| 1. | Glasgow outcome scale (GOS) |
| 2. | Barthel Index (BI) |
| 3. | Modified Rankin Score (MRS) |

### Age Distribution

| | STUDY GROUP | | | | | | | |
|---|---|---|---|---|---|---|---|---|
| | MS | | MT | | MT + MS | | CONTROL | |
| AGE | No. | % | No. | % | No. | % | No. | % |
| >50 | 0 | 0.00 | 0 | 0.00 | 0 | 0.00 | 0 | 0.00 |
| 51-55 | 5 | 33.33 | 3 | 20.00 | 3 | 20.00 | 1 | 6.67 |
| 56-60 | 5 | 33.33 | 2 | 13.33 | 5 | 33.33 | 6 | 40.00 |
| 61-65 | 3 | 20.00 | 5 | 33.33 | 3 | 20.00 | 6 | 40.00 |
| 66-70 | 1 | 6.67 | 3 | 20.00 | 3 | 20.00 | 1 | 6.67 |
| >70 | 1 | 6.67 | 2 | 13.33 | 1 | 6.67 | 1 | 6.67 |
| TO- | 15 | 100 | 15 | 100 | 15 | 100 | 15 | 100 |
| TAL | | | | | | | | |

| | | | | |
|---|---|---|---|---|
| Mean | 59.00 | 62.47 | 61.13 | 61.47 |
| Age | | | | |
| SD | 6.18 | 6.84 | 5.88 | 4.63 |

The mean age for magnesium sulfate (MS) group was 59.00 years; for mannitol (MT) group was 62.47 years; for magnesium sulfate plus mannitol (MT+MS) was 61.13 years and for the control group was 61.47 years. There is no significant difference between the age of study and control group.

### Sex Distribution

| | STUDY GROUP | | | | | | | |
|---|---|---|---|---|---|---|---|---|
| | MS | | MT | | MT + MS | | CONTROL | |
| SEX | No. | % | No. | % | No. | % | No. | % |
| MALE | 12 | 80.00 | 11 | 73.33 | 11 | 73.33 | 12 | 80.00 |
| FEMALE | 3 | 20.00 | 4 | 26.67 | 4 | 26.67 | 3 | 20.00 |
| TOTAL | 15 | 100 | 15 | 100 | 15 | 100 | 15 | 100 |

The sex distribution for magnesium sulfate group was 80.00 percent male; for mannitol group was 73.33 percent male; for magnesium sulfate plus mannitol was 73.33 percent male and for the control group was 80.00 percent male. There is no significant difference in the distribution of male female ratio between various groups.

### Risk Factors

| | STUDY GROUP | | | | | | | |
|---|---|---|---|---|---|---|---|---|
| | | | | | MT + | | | |
| RISK | MS | | MT | | MS | | CONTROL | |
| FACTOR | No. | % | No. | % | No. | % | No. | % |
| DM | 10 | 66.67 | 11 | 73.33 | 10 | 66.67 | 10 | 66.67 |
| HTN | 12 | 80.00 | 12 | 80.00 | 12 | 80.00 | 12 | 80.00 |
| AF | 3 | 20.00 | 3 | 20.00 | 2 | 13.33 | 2 | 13.33 |
| S. CHOLES- | 3 | 20.00 | 4 | 26.67 | 3 | 20.00 | 2 | 13.33 |
| TEROL >200 | | | | | | | | |
| MI | 2 | 13.33 | 2 | 13.33 | 2 | 13.33 | 1 | 6.67 |

Incidence of hypertension (HTN) was 80 percent in all the groups. The incidence of diabetes (DM) for magnesium sulfate group was 66.67 percent; for mannitol group was 73.33 percent; for magnesium sulfate plus mannitol was 66.67 percent and for the control group was 66.67 percent. There is no significant difference in the incidence of hypertension or diabetes mellitus in various groups. Incidence of myocardial infarction (MI) was 13.33 or less percent in all the groups. AF is atrial fibrillations and S. Cholesterol>200 means serum cholesterol is more than 200 mg/dl.

Glasgow outcome scale (GOS), Barthel Index (BI) and Modified Rankin Score (MRS) were compared in various groups and summarized as follows:

(a) Glasgow outcome scale (GOS), Barthel Index (BI), and Modified Rankin Score (MRS) were similar in control (patient not receiving magnesium or mannitol) group and mannitol group when measured on day 1, day 5, day 15, and day 30 of treatment. These results indicate that patients receiving mannitol were not improved compared to control patients who received neither magnesium sulfate nor mannitol. Therefore, mannitol was not effective compared to control.

(b) When a comparison was performed between the magnesium sulfate (16 gm) group and the mannitol group, it was observed that Glasgow outcome scale (GOS) was significantly lower in the mannitol group compared to magnesium sulfate (16 gm) group when measured at day 5, day 15, and day 30 of treatment. Barthel Index (BI) and Modified Rankin Score (MRS) were found to be significantly higher in the mannitol group compared to magnesium sulfate (16 gm) group when measured on day 5, day 15, and day 30 of treatment. These results indicate that patients treated with magnesium sulfate (16) group were improved compared to the mannitol (16 gm) group.

(c) When the comparison was performed between magnesium sulfate (8 gm) plus mannitol group and the mannitol group, it observed that Glasgow outcome scale (GOS) was significantly lower in the mannitol group compared to the magnesium sulfate (8 gm) plus mannitol group when measured at day 5, day 15, and day 30 of treatment. Barthel Index (BI) and Modified Rankin Score (MRS) were found to be significantly higher in the mannitol group compared to magnesium sulfate (8 gm) plus mannitol group when measured on day 5, day 15 and day 30 of treatment. These results indicate an improvement in patients treated with magnesium sulfate (8 gm) plus mannitol group compared to mannitol (16 gm) group.

(d) Glasgow outcome scale (GOS), Barthel Index (BI), and Modified Rankin Score (MRS) were similar in the magnesium sulfate (16 gm) group and the magnesium sulfate (8 gm) plus mannitol group when measured at day 1, day 5, day 15, and day 30 of treatment. These results indicate that recovery in patients in the magnesium sulfate (16 gm) group is similar to patients in magnesium sulfate (8 gm) plus mannitol group.

Evaluation of therapy for side effects of magnesium sulfate with and without mannitol treatment

| | Number out of total | Number out of total 15 |
|---|---|---|
| | 15 patients treated | patients treated with |
| | with magnesium | magnesium sulfate (8 gm) |
| SIDE EFFECTS | sulfate (16 gm) | plus mannitol group |
| Hypotension | 3 | 1 |
| Nausea | 5 | 2 |
| Constipation | 4 | 3 |
| Paradoxical | 3 | 3 |
| bradycardia | | |
| Heart block | 3 | 1 |
| Facial flushing | 8 | 4 |
| Respiratory | 2 | 1 |
| distress | | |

The incidence of side effects was greater in patients treated with magnesium sulfate (16 gm) alone compared to patients treated with magnesium sulfate (8 gm) plus mannitol group.

The results show that magnesium sulfate improves the treatment of patients with cerebrovascular accidents. It was found that magnesium sulfate (16 gm) was equally effective in patients that received magnesium sulfate (8 gm) plus mannitol. However, the incidence of side effects was higher in patients receiving magnesium sulfate (16 gm). From these results, it can be concluded that a combination of magnesium sulfate with mannitol improves the treatment of patients suffering from cerebrovascular accidents and traumatic brain injuries.

While the present invention is described in connection with specific examples, it should be appreciated that the invention is not limited to the disclosed examples, and is intended to cover various modifications and equivalent arrangements included within the spirit and scope of the claims. Modifications and variations in the present invention may be made without departing from the novel aspects of the invention as defined in the claims. It is understood that, given the above description of the examples of the invention, various modifications may be made by one skilled in the art. Such modifications are intended to be encompassed by the claims below.

Standard molecular biology protocols known in the art not specifically described herein are generally followed essentially as in Sambrook et al., “Molecular cloning: A laboratory manual,” Cold Springs Harbor Laboratory. New-York (1989, 1992), and in Ausubel et al., “Current Protocols in Molecular Biology,” John Wiley and Sons. Baltimore. Md. (1988).

Standard medicinal chemistry methods known in the art not specifically described herein are generally followed essentially as in the series “Comprehensive Medicinal Chemistry,” by various authors and editors, published by Pergamon Press.

The success of magnesium in attenuating the process of neuro-degeneration in animal models of brain injury has been widely studied. However, the preclinical studies have not been translated into successful clinical outcome. In TBI patients, administration of magnesium has shown variable results. Secondary brain insults and other parameters adversely affect the clinical outcome, which could have unfavorably influenced the result of the efficacy studies with magnesium in TBI patients. In the design of clinical studies on brain injuries, secondary brain insults and parameters adversely affecting outcome need to be considered.

### REFERENCES

1. N. R. Temkin et al. Lancet Neurol. 2007; 6: 29-38.
2. S. S. Dhandapani et al. Indian J Neurotrauma. 2008; 5: 27-33.
3. M. Hallak et al. Am J Obstet Gynecol. 1992; 167: 1605-10.
4. M. R. Hoane. Magnes Res. 2007; 20: 229-36.
5. Fuchs-Ruder et al. J Neurosurg Anesthesiol. 1997; 9: 324-8.
6. J. A. McKee et al. Crit Care Med. 2005; 33: 661-6.
7. G. R. Thurnau. Am J Obstet Gynecol. 1987; 157: 1435-8.
8. B. Stoica. Neurotherapeutics. 2009; 6: 14-27.
9. R. Vink. Neurotherapeutics. 2009; 6: 28-42.
10. N. Stocchetti et al. J Neurotrauma. 2007; 24: 1339-46.
11. M. Balestreri et al. Neurocrit Care. 2006; 4: 8-13.
12. G. Fiskum. J Neurotrauma. 2000; 17: 843-55.
13. J. Lifshitz et al. J Cereb Blood Flow Metab. 2003; 23: 219-31.
14. C. P. Hans et al. Indian J Exp Biol. 2002; 40: 1275-9.
15. L. A. Garcia et al. J Am Coll Cardiol. 1998; 32: 536-9.
16. I. Cernak et al. J Neurotrauma. 2000; 17: 53-68.
17. M. T. Chan et al. Acta Neurochir Suppl. 2005; 95: 107-11.
18. J. A. Napieralski et al. Brain Res Mol Brain Res. 1999; 71: 78-86.
19. J. S. Lee et al. J Neurotrauma. 2004; 21: 549-61.
20. S. Nielsen et al. J Neurosci. 1997; 17: 171-80.
21. M. N. Ghabriel et al. Acta Neurochir Suppl. 2006; 96: 402-6.
22. K. Okiyama et al. J Neurochen. 1995; 64: 802-9.
23. D. L. Heath et al. J Clin Neurosci. 1999; 6: 505-9.
24. F. M. Bareyre et al. J Neurochem. 73: 271-80.
25. C. van den Heuvel et al. Clin Calcium. 2004; 14: 9-14.
26. Z. Feldman et al. J Neurosurg. 1996; 85: 131-7.
27. K. D. Browne et al. J Neurosci Res. 2004; 77: 878-83.
28. M. R. Hoane. Clin Calcium. 2004; 14: 65-70.
29. M. R. Hoane et al. Brain Res Bull. 2003; 60: 105-14.
30. Heath D L et al. J Pharmacol Exp Ther. 1999; 288: 1311-6.
31. F. M. Bareyre et al. J Neurotrauma. 2000; 17: 1029-39.
32. D. L. Heath. J Neurosurg. 1999; 90: 504-9.
33. M. R. Hoane et al. Physiol Behav. 2002; 76: 271-80.
34. S. Canavero et al. Surg Neurol. 2003; 60: 165-9.
35. J. E. Natale et al. Pediair Crit Care Med. 2007; 8: 1-9.
36. T. Sakamoto et al. J Trauma. 2005; 58: 1103-9.
37. A. M. Kafadar et al. Neurol Res. 2007; 29: 824-9.
38. R. Schmid-Elsaesser et al. Neurosurgery. 1999; 44: 163-71.
39. A. B. Barbre et al. Brain Res Bull. 2006; 69: 639-46.
40. N. Knoller et al. Crit Care Med. 2002; 30: 548-54.
41. C. A. et al. Brain Res. 2005; 1062: 171-4.
42. E. H. Pettus et al. Brain Res. 2005; 1049: 112-9.
43. H. Zhu et al. Exp Neurol. 2005; 193: 361-8.
44. K. Campbell et al. Brain Res. 2008; 1230: 258-64.
45. R. Schmid-Elsaesser et al. Stroke. 1999; 30: 1891-9.
46. S. Zausinger et al. Stroke. 2003; 34: 2246-51.

What is claimed is:

1. A pharmaceutical composition comprising a magnesium salt and a hypertonic osmotic agent.

2. The composition of claim 1 wherein the magnesium salt comprises magnesium sulfate, magnesium chloride, or a mixture thereof.

3. The composition of claim 1 wherein the hypertonic osmotic agent is selected from the group consisting of mannitol, hypertonic saline (2.5-10% NaCl), hypertonic saline with dextran, hypertonic saline with hetastarch, dextran (5 to 50%), inulin, hetastarch, pentastarch, urea, glycerol, arabinose, sucrose, lactamide, and a mixture thereof.

4. The composition of claim 1 wherein the magnesium salt is magnesium sulfate.

5. The composition of claim 1 wherein the magnesium salt is magnesium chloride.

6. The composition of claim 1 wherein the hypertonic osmotic agent comprises mannitol, hypotonic saline, or a mixture thereof.

7. The composition of claim 1 wherein the ratio of the magnesium salt to the hypertonic osmotic agent is about 1:0.1 to about 1:100.

8. The composition of claim 7 wherein the ratio of the magnesium salt to the hypertonic osmotic agent is about 1:0.5 to about 1:10.

9. A method of treating an individual suffering from a neurological insult comprising administration to the individual a therapeutically effective amount of a magnesium salt and a therapeutically effective amount of a hypertonic osmotic agent.

10. The method of claim 9 further comprises administering at least one additional therapeutic agent useful in a treatment of the neurological insult.

11. The method of claim 10 wherein the at least one additional therapeutic agent comprises a pharmacological agent, a physiological agent, or both.

12. The method of claim 11 wherein the pharmacological agent is selected from the group consisting of tirilazad, vitamin B, riboflavin, dexanabinol, progesterone, a statin, progestereone, erythropoietin, minocycline, a Toll-like receptor agonist, dexanabinol, a thyrotropin releasing hormone analog, cyclosporin-A, and mixtures thereof.

13. The method of claim 11 wherein a physiological agent comprises hypothermia, hypoxia, or both.

14. The method of claim 9 wherein the neurological insult is selected from the group consisting of traumatic brain injury, increased intracranial pressure, brain edema due to head injury, intoxication, hepatic failure, a space-occupying cerebral lesion, meningitis, Reye's syndrome, cerebral malaria, a brain tumor, birth asphyxia, perinatal asphyxia, asphyxiated neonate asphyxiated infant, rebound phenomenon in the treatment of raised intracranial pressure, hyperglycemic crisis and resulting complications, diabetic ketoacidosis, acute stroke, ischemic stroke, cerebral hemorrhage, focal ischemia, subarachnoid hemorrhage, drug induced hypomagnesaemia, a neurological complication of a chemotherapeutic agent, preeclampsia, an epileptic episode, prolongation of opiate and non-opiate analgesia, an affective disorder, post-traumatic depression/anxiety, a neuropsychiatric disorder, a headache, a migraine, and neuroprotection of an adult or neonatal brain.

15. The method of claim 10 wherein magnesium salt, the hypertonic osmotic agent, and the second therapeutic agent are administered simultaneously.

16. The method of claim 10 wherein the magnesium salt and the hypertonic osmotic agent are administered simultaneously, and the second therapeutic agent is administered separately.
